(19) United States
(12) Patent Application Publication
(10) Pub. No.: US 2017/0209086 A1
(43) Pub. Date: Jul. 27, 2017

(54) PORTABLE DEVICE FOR MEASURING PHYSICAL PARAMETERS ASSOCIATED WITH THE ANKLE JOINT OF A SUBJECT, AND FRAME OF SUCH A DEVICE

(71) Applicants: UNIVERSITE DE TECHNOLOGIE DE COMPIEGNE, Compiegne (FR); CENTRE NATIONAL DE LA RECHERCHE SCIENTIFIQUE, Paris (FR)

(72) Inventors: Francis CANON, La Croix Saint-Ouen (FR); Didier GAMET, Villeparisis (FR)

(73) Assignees: UNIVERSITE DE TECHNOLOGIE DE COMPIEGNE, Compiegne (FR); CENTRE NATIONAL DE LA RECHERCHE SCIENTIFIQUE, Paris (FR)

(21) Appl. No.: 15/515,232

(22) PCT Filed: Sep. 28, 2015
(86) PCT No.: PCT/EP2015/072205

§ 371 Date: Mar. 29, 2017

(30) Foreign Application Priority Data
Oct. 1, 2014 (FR) 14 59338

(51) Int. Cl. A61B 5/00; A61B 5/11

(52) U.S. Cl. CPC A61B 5/4595; A61B 5/4528; A61B 5/45; A61B 5/1121; A61B 5/11; A61B 5/702; A61B 5/00

(57) ABSTRACT

The invention relates to a portable device (1) comprising a frame (2) which comprises a receptacle (9) that is intended to receive a subject's foot and is mounted in a pivoting manner on the frame, a means (10, 13) for temporarily locking the receptacle in a first position, the device comprising means (19) for determining a torque exerted on the receptacle, means (20) for determining an angular displacement of the receptacle relative to the frame, and means (22) for determining an angular acceleration of the receptacle during an angular displacement of the receptacle, at least the locking means and the means for determining a torque being arranged in the frame behind the receptacle when the receptacle opening faces the subject. The invention also relates to a frame of such a device.

The invention relates to a portable device for measuring physical parameters associated with the ankle joint of a subject, in particular of a patient. The invention also relates to a frame of such a device.

### TECHNICAL BACKGROUND OF THE INVENTION

The study of physical parameters associated with the ankle joint, more precisely those associated with the effector muscles allowing movement of the ankle joint, is of interest in many areas. In the medical field, of course, it proves important to understand the functioning and evolution of these effector muscles. This is all the more of interest as regards bedridden patients. During bed rest, muscle properties such as contractility and/or elasticity (maximum range of motion of the ankle in plantar flexion and dorsiflexion, stiffness of the ankle depending on its angular position, maximum voluntary torque developed, index of musculotendinous stiffness, index of neuromuscular efficiency, etc.) may be caused to change. However, the study of physical parameters related to the ankle of a patient also proves important in other fields such as sports, with a view to improving athlete performance.

Therefore, various tests are known for measuring physical parameters associated with the ankle joint of a subject, for example the test for measuring the index of musculotendinous stiffness of the ankle, better known as the “quick release” test. This test involves placing the subject on a measuring apparatus comprising a study device with a pedal and asking the subject to press down as firmly as possible on said pedal, the pedal being kept fixed before being suddenly released during the test. Various sensors connected to the measuring apparatus then allow data to be recorded during this test, for example the force exerted on the pedal by the subject, the angular displacement of the pedal when it is released, etc.

However, this type of measuring apparatus proves very bulky and, when the subject is placed on such an apparatus with his foot on the pedal, the foot and the ankle may be partially or completely concealed by the study device, by wires linking the device to the various sensors, etc.

The measuring apparatus thus proves very daunting for many subjects, especially for children, who then struggle to relax when the “quick release” test is carried out. This tends to distort the test results, which is not desirable.

Moreover, a measuring apparatus of this kind cannot be used for carrying out measurements on subjects who have difficulty walking or are unable to move, since it then proves extremely difficult to place these subjects on the apparatus.

### OBJECT OF THE INVENTION

It is an object of the invention to make available a device for measuring physical parameters associated with the ankle joint of a subject, even if the subject has difficulties moving, said device permitting more meaningful measurements of said physical parameters, and also a frame of such a device.

### BRIEF DESCRIPTION OF THE INVENTION

To achieve this object, a portable device for measuring physical parameters associated with the ankle joint of a subject is made available, said device having:
a portable frame which has an open receptacle intended to receive a foot of the subject, the receptacle being mounted pivotably on the frame about a given rotation axis in such a way as to be arranged at the end of the frame with an opening of the receptacle directed toward the outside of the device and in such a way that the rotation axis coincides with the articulation axis of the subject's ankle when the subject's foot rests in the receptacle, and which has means for temporarily locking the receptacle in a first position,
means for determining a torque exerted on the receptacle,
means for determining an angular displacement of the receptacle relative to the frame,
means for determining an angular acceleration of the receptacle during an angular displacement of the receptacle,
   at least the locking means and the means for determining a torque being arranged in the frame behind the receptacle when the opening of the receptacle faces the subject.

By virtue of the design of the receptacle, the subject basically only sees the frame and the receptacle during the tests, most of the elements carried by the frame being concealed from the subject by the receptacle itself.

The frame according to the invention thus proves to be compact and has a simplified structure, which makes it look more reassuring to a subject. The frame ensures in particular that, during the tests, the foot and ankle are not concealed by components of the device, other than optional means for immobilizing the foot on the receptacle, which concealment tended to frighten some subjects, especially children.

By virtue of the device according to the invention, subjects are able to feel more relaxed during the tests, allowing more meaningful test results to be obtained.

In addition, it proves easier to carry the device according to the invention, particularly on account of the reduced volume and simplified structure of the frame.

Moreover, apart from the fact that the patient's foot has to be placed in the open receptacle, the device according to the invention by its very design, thanks to its portable frame, does not restrict the position of the subject. Indeed, the size and weight of the frame are adapted to its being handled by one person and placed at locations suitable for the subjects, without the subjects having to change position.

The device according to the invention thus proves particularly advantageous for studying physical parameters related to the ankle joints of bedridden subjects, in particular bedridden patients. This is because it is possible to place the frame of the device according to the invention on a panel arranged astride the patient's bed, such that the tests can be carried out without having to lift or move the patient. All the patient has to do is stretch out his leg in order to place his foot in the receptacle.

This is all the more interesting given that studying the behavior of the ankle of a bedridden patient proves particularly important since, during bed rest, the muscle properties of the ankle, such as contractility and/or elasticity (maximum range of motion of the ankle in plantar flexion and dorsiflexion, stiffness of the ankle depending on its angular position, maximum voluntary torque developed, index of musculotendinous stiffness, index of neuromuscular efficiency, etc.), may be caused to change.

Furthermore, the device according to the invention can be easily carried from one bed to another, again by virtue of its portable frame.

Moreover, by virtue of the open receptacle and the fact that the frame can be moved relative to the position of the subject, the device can therefore be used both for the left ankle and for the right ankle. The device is thus designated as ambidextrous. The same device can thus be used by a large number of subjects.

In the present application, means for determining are understood as measuring means permitting a physical measurement of the parameter in question, calculating means permitting an estimation, by mathematical calculation, of the parameter in question, and also a combination of measuring means and calculating means.

The invention also relates to a portable frame of a device as described above, the frame having an open receptacle intended to receive a foot of the subject, the receptacle being mounted pivotably on the frame about a given rotation axis in such a way as to be arranged at the end of the frame with an opening of the receptacle directed toward the outside of the device and in such a way that the rotation axis coincides with the articulation axis of the subject's ankle when the subject's foot rests in the receptacle, the frame having means for temporarily locking the receptacle in a first position.

### BRIEF DESCRIPTION OF THE DRAWINGS

The invention will be better understood in light of the following description of a particular non-limiting embodiment of the invention. Reference will be made to the attached figures, in which:

FIG. 1 is a schematic view showing a bedridden subject, with a portable device according to the invention arranged astride the subject's bed,

FIG. 2 is a perspective view of part of the device illustrated in FIG. 1, the receptacle of said device being in its first position, the side walls of the frame being shown by broken lines,

FIG. 3 is a perspective view of part of the device illustrated in FIG. 2, the receptacle of said device being in its second position, the side walls of the frame being shown by broken lines,

FIG. 4 is a perspective view identical to that of FIG. 2, in which only the frame and the associated housings are shown,

FIG. 5 is a perspective view identical to that of FIG. 2, in which the side walls of the frame are not shown.

### DETAILED DESCRIPTION OF THE INVENTION

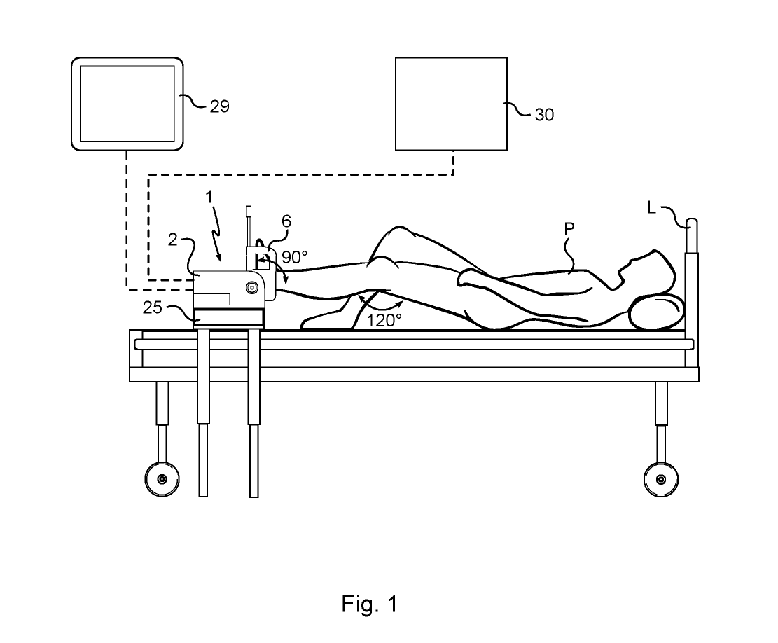

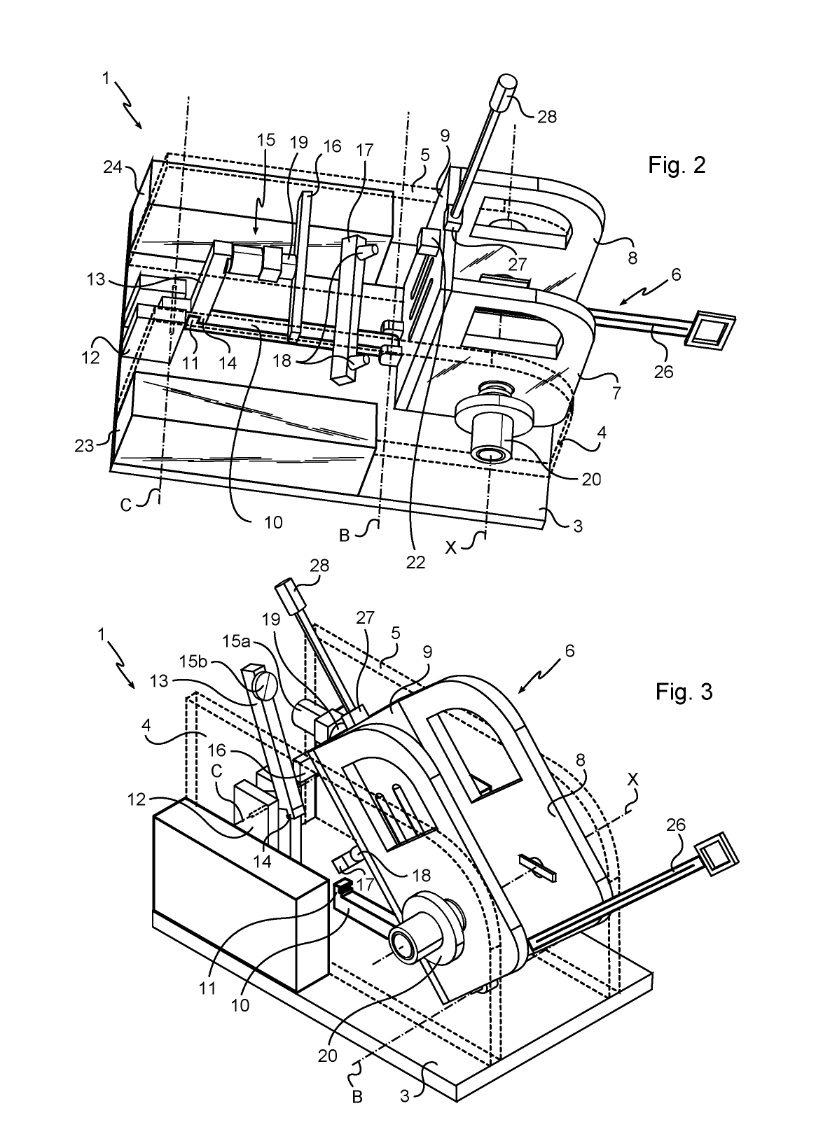

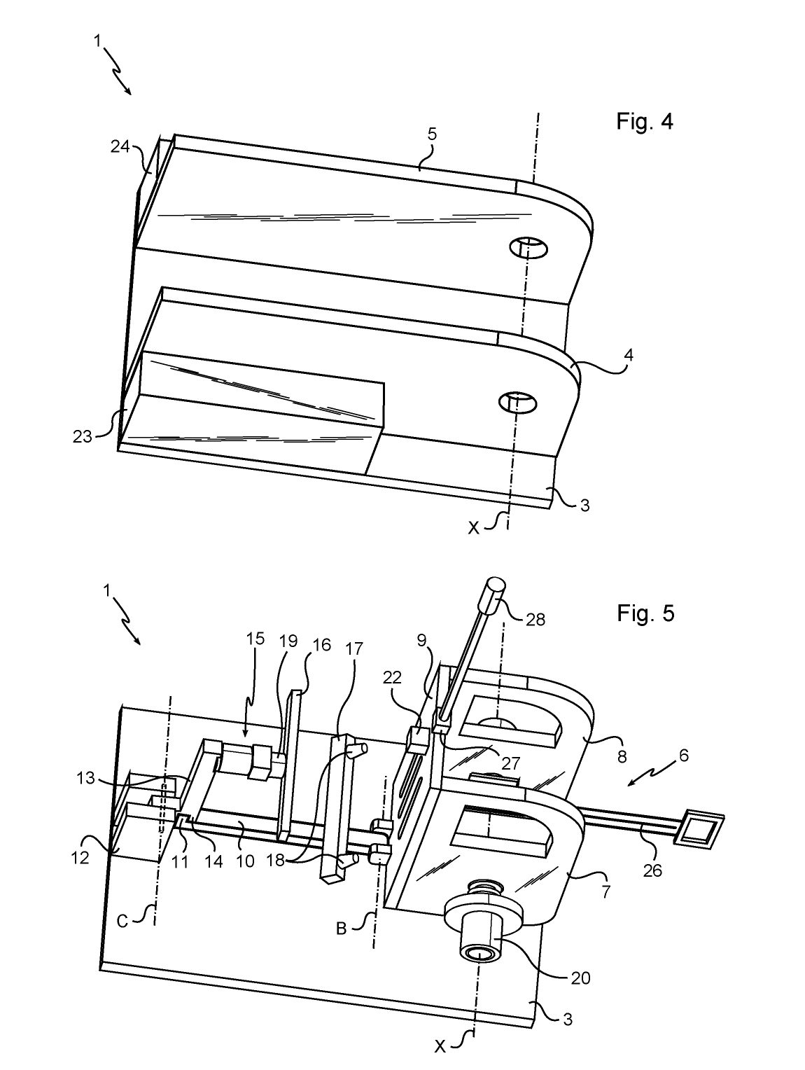

With reference to FIGS. 1 to 5, the portable device 1 according to the invention for measuring physical parameters associated with the ankle joint of a subject, here a patient P, comprises a portable frame 2. The frame 2 here comprises a floor 3, and two side walls 4, 5 extending in parallel from the floor 3 so as to face each other.

The frame 2 additionally has an open receptacle 6 intended to receive a foot of the patient P. Here, the receptacle 6 has substantially the shape of a bracket with two wings 7, 8 and a base from which the two wings 7, 8 extend. The base forms a main wall 9 of the receptacle 6, on which wall the sole of the patient P is intended to rest. The first wing 7 is mounted pivotably on the first side wall 4 about a rotation axis X, and the second wing 8 is mounted pivotably on the second side wall 5 about said rotation axis X. Thus arranged, the receptacle 6 extends between the two side walls 4, 5 so as to be mounted pivotably about the same rotation axis X. The receptacle 6 is additionally arranged at the end of the frame 2 with an opening of the receptacle 6 directed toward the outside of the frame 2.

Thus, when the patient P looks at the frame 2, with the opening of the receptacle 6 facing toward him, everything contained between the two side walls 4, 5 is behind the receptacle 6 and is therefore concealed from the patient P by the receptacle 6.

The receptacle 6 is additionally arranged in such a way that the rotation axis X coincides with the axis of articulation of the ankle of the patient P when his foot rests in the receptacle 6. This ensures that the ankle will be able to turn correctly during the measurements, thereby allowing meaningful measurement results to be obtained.

To avoid the patient's foot slipping or accidentally moving when the measurements are being taken, which would distort the results, the frame 2 has means for immobilizing the foot on the receptacle 6. The immobilizing means comprise a strap 26, for example.

Furthermore, the frame 2 has locking means for locking the receptacle 6 in a first position.

Typically, the locking means have a first lever 10, which is pivotably mounted at its first end on the receptacle 6. More exactly here, the first lever 10 is mounted pivotably about an axis B parallel to the rotation axis X of the receptacle 6 at the lower end of the main wall 9. The first lever 10 has a hook 11 at its second free end. The frame 2 here has a panel 12 arranged between the two side walls 4, 5 at one end of the floor 3 opposite the end where the receptacle 6 is arranged. The locking means additionally have a second lever 13, of which a first end is mounted pivotably on said panel 12 along an axis C parallel to the rotation axis X of the receptacle 6. The first end additionally has a hook 14 adapted to cooperate with the hook 11 of the first lever 10 in order to lock the receptacle 6 in the first position.

According to a particular embodiment of the invention, the frame 2 has a member 15 for activating the locking means in such a way that, when the activating member 15 is not powered, the locking means are active and, when the activating member 15 is powered, the locking means are not active and do not lock the receptacle 6, which is able to pivot relative to the frame 2. The activating member 15 is of the electromagnetic type, for example. The frame 2 here has a first bar 16 extending transversely between the two side walls 4, 5, so as to be rigidly fixed at each of its ends to a respective one of the side walls 4, 5. A first portion 15a of the activating member 15 is integral with said first bar 16 and is here arranged substantially at the center of said first bar 16. A second portion 15b of the activating member 15 is integrally connected to the second end of the second lever 13.

Thus, when the activating member 15 is not powered, the two portions of the activating member 15 attract each other, so as to be integral with each other. In this position, the second lever 13 extends parallel to the two side walls 4, 5 between the first portion 15a and the panel 12, the second end of the second lever 13 being rigidly connected to the frame 2 via the activating member 15. The first lever 10 extends between the receptacle 6 and the second lever 13 and substantially perpendicularly with respect to the first lever 13, the two hooks 11, 14 cooperating with each other such that the first lever 10 is rigidly connected to the frame 2 via the second lever 13. This defines the first position of the receptacle 6. The locking means are arranged in such a way that, in the first position, the main wall 9 of the receptacle 6 extends perpendicularly with respect to to the floor 3.

When the activating member 15 is powered, the two portions of the activating member 15 separate from each other, thus freeing the rotation of the second lever 13 about the panel 12 along the axis C and, consequently, separating the two levers 10, 13. In this way, the rotation of the first lever 10 about the receptacle 6 along the axis B is permitted, which causes a rotation of the receptacle 6 about its rotation axis X. By powering the activating member 15, it is thus possible to make the receptacle 6 tilt from the first position to a second position.

Advantageously, the device 1 does not use much power since the activating member 15 keeps the first portion 15a and the second portion 15b integral with each other as long as it is not powered. A simple electrical pulse is enough to temporarily disable the activating member 15 and allow the receptacle 6 to tilt from the first position to the second position. Said pulse is here less than a second, which makes the device 1 particularly efficient in terms of power consumption.

To tilt the receptacle 6 from the second position to the first position and again keep the receptacle 6 locked in the first position, it is necessary to reset the device 1, which can be done very easily, for example by a member of the medical profession, or possibly by the patient P himself, in a movement of dorsiflexion of the ankle.

The frame 2 also has means for limiting an angular course of the receptacle 6 from the first position to the second position. For this purpose, the limiting means here have a second bar 17 extending transversely between the two side walls 4, 5 such that the main wall 9 abuts the second bar 17 when the receptacle 6 is in its second position. Said means for limiting the angular course of the receptacle 6 are preferably removable, so that for some tests, as we will see below, it is possible not to limit the angular course of the receptacle 6 and, therefore, the plantar flexion of the ankle. The second bar 17 is therefore removable here.

In a particular embodiment, when the second bar 17 is arranged on the frame, the second bar 17 is arranged in such a way that, in the second position, the main wall 9 extends a maximum of 45 degrees from the floor 3. This maximum value of 45 degrees corresponds to an estimation of the maximum plantar flexion that proves non-traumatic for most subjects. Here, the second bar 17 is arranged in such a way that, in the second position, the main wall 9 extends substantially at 45 degrees from the floor 3.

The frame 2 is thus arranged in such a way that the first position corresponds to a locked configuration of the receptacle 6, in which the foot extends at 90 degrees from the tibia, and in such a way that the second position corresponds to an unlocked configuration of the receptacle 6, in which the foot extends substantially 135 degrees from the tibia, that is to say in which the foot is at its maximum plantar flexion that proves non-traumatic to the patient P. Preferably, the second bar 17 comprises pegs 18 made of elastically deformable material, for example of rubber, in order to cushion the fall of the receptacle 6 from the first position to the second position.

Furthermore, the frame 2 has means for determining a torque exerted on the receptacle 6 by the foot of the patient P. To this end, the means for determining a torque here comprise a first force sensor 19. Said first force sensor 19 is arranged, for example, at the center of the first bar 16 in the continuation of the first portion 15a of the activating member 15. This makes it possible to measure the force exerted on the receptacle 6 by the foot of the patient P, when the receptacle 6 is in its first position, via the first lever 10, the second lever 13 and the activating member 15. By multiplying said measured force by a coefficient taking account of the lever arm lengths (that is to say the orthogonal distance to the force between the hook 11 and the axis C, the orthogonal distance to the force between the axis B and the axis X, and the orthogonal distance to the force between the first force sensor 19 and the axis C), the determining means determine the torque exerted on the receptacle 6 by the foot of the patient P.

According to a particular embodiment, the means for determining a torque here comprise a second force sensor 27. Said second force sensor 27 is here arranged at an upper end of the main wall 9, opposite a lower end of the main wall 9 facing the floor 3. The means for determining a torque here comprise a handle 28 which is rigidly connected to the second force sensor 27. Here, the handle 28 thus extends outward from the frame substantially vertically when the receptacle 6 is in its first position.

This makes it possible to determine a passive torque exerted on the receptacle 6 by the foot of the patient P when the receptacle 6 is moved manually by a member of the medical profession.

The frame 2 additionally has means for determining an angular displacement of the receptacle 6 relative to the frame 2. For this purpose, the means for determining an angular displacement comprise means for measuring an angular displacement of the receptacle 6. Here, the measuring means comprise an angular displacement sensor 20 arranged on the first side wall 4 at the level of the rotation axis X.

The frame 2 moreover has means for determining an angular acceleration of the receptacle 6 during an angular displacement of the receptacle 6 relative to the frame 2. For this purpose, the means for determining an angular acceleration comprise an accelerometer 22 arranged on a face of the main wall 9 opposite the one intended to receive the sole of the foot.

Preferably, the frame 2 has means for measurement of electromyographic signals. Such means comprise, in a manner known per se, non-invasive surface electrodes (not shown here) to be arranged in the area of the muscles mobilizing the ankle of the patient P.

According to a preferred embodiment, the locking means, the activating member 15, the force sensors 19 and 27, the accelerometer 22 and the means for limiting the angular course are arranged in such a way as to be concealed by the receptacle 6 when the receptacle opening faces the patient P. Only the angular displacement sensor 20, the electrodes and, in one embodiment, the handle 28 are thus visible to the patient P during the tests. However, this does not obstruct the view of the patient P, who is still able to see his foot in the receptacle only covered by the strap 26.

The frame 2 is therefore compact and simple in appearance, which is reassuring to the patient P.

Preferably, the locking means, the activating member 15, the force sensors 19 and 27, the handle 28, the accelerometer 22, the means for limiting the angular course and the angular displacement sensor 20 extend here at a height lower than that of the receptacle 6 when the receptacle 6 is in its first position.

This makes the frame 2 less bulky. In addition, the receptacle 6 remains the largest part of the frame 2. This reassures the patient P still further.

The frame 2 also has management means for managing the force sensors 19 and 27, the angular displacement sensor 20, the accelerometer 22, the means for measurement of electromyographic signals and the activating member 15. The management means advantageously have means for conditioning and processing the signals generated by the force sensors and 27, the angular displacement sensor 20, the accelerometer 22 and the electrodes, in order to supply data usable by the medical profession.

The management means further comprise wireless communication means, for example by a Wi-Fi or Bluetooth protocol. The device additionally has a remote control unit. The wireless communication means communicate here with said remote control unit.

The remote control unit makes it possible, in the first instance, to receive said data usable by the medical profession in order to complete the processing and save the data, signals and results, and, in the second instance, to send commands to the management means in order to manage the various study tests.

The control unit is here a tablet 30. Alternatively, the control unit may be a computer, a smartphone or even a microcomputer.

The frame 2 moreover has autonomous means for supply of power to the frame 2, comprising a battery, for example, which can be rechargeable.

This makes the frame 2 highly autonomous. In addition, the frame 2 does not need to be connected to the mains, which eliminates the need for an associated isolation amplifier. It will be recalled that, in the medical field, such an amplifier is obligatory when a medical device is used by a patient and is connected to the mains.

The management means and the battery are here placed in two housings 23, 24 which are rigidly connected to the floor 3 and which are arranged on either side of the side walls 4, 5. The management means, the battery and the two housings 23, 24 are preferably arranged set back from the receptacle 6 when the patient P has the receptacle 6 facing toward him. This allows the receptacle to remain the feature that is most visible to the patient P.

The frame 2 thus proves fully autonomous, in particular by virtue of the battery and the wireless communication means.

In addition, the frame 2 has a very small volume and is of a simple appearance. The inventors have thus been able to develop a frame 2 whose floor is less than 500 millimeters in length and less than 300 millimeters in width with a maximum height (between the floor and the receptacle when the receptacle is in its first position) of less than 400 millimeters.

The frame 2 is therefore of a size and weight adapted to its being handled by just one person and to being placed at locations suitable for subjects, without the subjects having to change position. It is not just that the frame 2 is portable but also that, by moving the frame, a large part of the device according to the invention is moved, since the frame 2 carries most of the determining means of the device.

The device 1 is therefore perfectly adapted to the study of physical parameters associated with the ankle joints of bedridden patients, since the frame 2 is sufficiently compact and ergonomic to be able to be placed on the patient's bed at the area of the patient's feet.

Preferably, the device 1 has a panel 25 which is here rigidly connected to the frame 2, the panel 25 being provided with feet that are adjustable in height.

In this way, it is possible to adjust the height of the frame 2, and therefore of the receptacle 6, with respect to the bed L of the patient P when the frame 2 is arranged on the panel 25, as is shown in FIG. 1. In the various tests for studying physical parameters related to the ankle joint, the leg must preferably be in a particular position in order to obtain more meaningful data with, when the receptacle 6 is in the first position, an angle of 90 degrees between the foot and the tibia and an angle of 120 degrees between the femur and the tibia. By virtue of the adjustable feet of the panel 25, it is ensured that the receptacle 6 is always arranged at a suitable height so that the leg of the patient P is in said particular position.

The device 1 has means for interfacing with the patient and/or the medical team. In addition to the aforementioned control unit, here a tablet 30, the interface means preferably comprise a screen 29 on which are indicated the data values acquired during the tests and/or test instructions which the patient has to follow and which are generated by the tablet 30.

Besides being of a limited volume and of an ergonomic and simplified configuration, by virtue of its frame 2 in particular, the device 1 permits the implementation of numerous tests for acquiring signals to determine various physical parameters associated with the ankle joint. For example, the device 1 allows the following tests to be carried out under conditions of voluntary muscular contraction or relaxation of the ankle joint by the patient P.

a) Test for Measuring Joint Flexibility and Passive Stiffness

The receptacle 6 is not kept locked by the locking means. The second bar 17 is removed so as not to limit the movement of the receptacle 6. The patient P simply places his foot in the receptacle 6 without exerting voluntary effort on the main wall 9. The means for measurement of electromyographic signals make it possible to estimate an absence of contraction of the ankle muscles of the patient P, signifying that the patient P is not pressing on the main wall 9.

In this case, a member of the medical profession, such as a physician, a physiotherapist, a person trained in kinesitherapeutic massage, etc., causes a movement of the ankle by manually moving the receptacle 6 slowly to the maximum plantar flexion and dorsiflexion tolerated by the patient P. To this end, the member of the medical profession uses the handle 28 to move the receptacle 6. It is then possible to determine the passive torque, developed by the patient P for following the movement of the receptacle 6, with the aid of the second force sensor 27, and simultaneously to determine the angular position of the receptacle, during the movement, with the aid of the angular displacement sensor 20, and to determine the electromyographic signals with the aid of the electrodes. This permits determination of the following parameters: maximum range of motion of the ankle in plantar flexion and dorsiflexion, and stiffness of the ankle depending on its angular position.

b) Test for Measuring the Maximum Voluntary Force that the Patient P is Able to Develop

The receptacle 6 is in its first position. The patient P places his foot in the receptacle 6 and presses as firmly as possible on the main wall 9 in a movement of plantar flexion. The torque developed by the patient P is then determined with the aid of the first force sensor 19, and the electromyographic signals are determined with the aid of the electrodes. This makes it possible to determine the following parameter: maximum voluntary torque developed by the patient P.

c) Test for Measuring the Musculotendinous Stiffness (or “Quick Release” Test)

The receptacle 6 is kept locked in its first position. The second bar 17 is in place on the frame 2. The patient P places his foot in the receptacle 6 and, in a movement of plantar flexion, presses on the main wall 9 in order to attain a constant torque level which is defined by a torque setpoint generated by the tablet 30 and which corresponds to a given percentage of the maximum voluntary torque calculated previously. Several setpoints are successively generated for the attention of the patient P. Typically, the patient P has to place his foot in the receptacle 6 to as to successively attain a constant torque level of 25, 50 and 75% of the maximum voluntary torque.

When the constant torque level is attained and is stable, an electrical pulse is transmitted to the activating member 15, which suddenly releases the locking means of the receptacle 6 such that the receptacle 6 suddenly tilts to its second position. The torque developed by the patient P is then determined with the aid of the first force sensor 19 a few tenths of a second before the receptacle 6 is unlocked. Moreover, the angular position of the receptacle 6 is determined with the aid of the angular displacement sensor 20, and the angular acceleration with the aid of the accelerometer 22, during the movement of the receptacle 6 from the first position to the second position, i.e. during the dynamic phase of the test. In addition, the electromyographic signals are recorded throughout the test with the aid of the electrodes.

Since the “quick release” test does not permit measurement of the torque developed by the patient P during the dynamic phase of the test, it is acknowledged that during the first moments of this phase, when acceleration is at its maximum, the dynamic torque is equal to the static torque developed by the patient P when the receptacle 6 is in the first position, the static torque which has thus been measured. Given that the dynamic torque is equal to the product of the inertia of the ankle by the maximum angular acceleration of the ankle, here the maximum angular acceleration of the receptacle 6 measured during the dynamic phase, the inertia is deduced by:

Inertia=Static torque measured before the release of the receptacle 6/Maximum angular acceleration measured during the dynamic phase

Moreover, for each level of torque developed by the patient P, the musculotendinous stiffness is calculated for a period of 20 milliseconds of dynamic phase after the acceleration peak following the release of the receptacle 6 (this duration is deliberately chosen to be short in order to ensure that the stretching reflex occurring in the antagonist muscles does not take place during said period, which would distort the results, the stretching reflex generally taking place after 30 milliseconds following the acceleration peak) by the formula:

Musculotendinous stiffness=Inertia. (Variation in angular acceleration over the period of 20 milliseconds/Variation in angular position over the period of 20 milliseconds)

Proceeding from the musculotendinous stiffness calculated for each level of torque developed by the patient P, it is possible to deduce the function linking musculotendinous stiffness and torque developed by the patient P. An index of musculotendinous stiffness is then evaluated by considering the slope of the linear relationship between musculotendinous stiffness and torque or electromyographic activity.

d) Test for Measuring Neuromuscular Efficiency

The receptacle 6 is kept locked in its first position. The patient P places his foot in the receptacle 6 and, in a movement of plantar flexion, presses on the main wall 9 in order to attain a constant torque level which is defined by a torque setpoint generated by the tablet 30 and which corresponds to a given percentage of the maximum voluntary torque calculated previously. Several setpoints are successively generated for the attention of the patient P. Typically, the P patient must place his foot in the receptacle 6 in order to successively attain a level of constant torque equal to 20, 40, 60 and 80% of the maximum voluntary torque.

When the constant torque level is attained and is stable, the torque developed by the patient P is then determined with the aid of the first force sensor 19, and the electromyographic signal is determined with the aid of the electrodes. Proceeding from the electromyographic signal for each level of torque developed by the patient P, it is possible to deduce the function linking said signal and the torque developed by the patient P. An index of neuromuscular efficiency is then evaluated by considering the slope of the linear relationship between electromyographic signal and torque.

Of course, the invention is not limited to the embodiment described and can be modified without departing from the scope of the invention as defined by the claims.

In particular, although the device is used here for studying physical parameters related to the ankle joint of a patient, the device will be able to be used for studying physical parameters related to the ankle joint of other categories of persons, in particular on subjects free of pathologies.

In addition, it will be possible for the device to have additional means for measuring physical parameters associated with the ankle joint of a subject, such as a neurostimulator, or an Achilles tendon percussion system comprising a pneumatic hammer, for example. The device will thus allow other tests to be carried out based, for example, on a contraction imposed on the muscles of the ankle by percussion with the percussion system or by electrical stimulation with the neurostimulator (test of muscle twitch test, Hoffmann reflex test, tendon reflex test, activation deficit test, etc.). The additional means will preferably also be carried by the frame, the additional means preferably not obstructing the subject's view of the receptacle so that he can at all times see his foot, possibly partially covered by the immobilizing means.

The means for measuring parameters associated with the ankle joint of the subject may be different than what has been described. It will be possible for the device not to have means for measurement of electromyographic signals. In the case where the device has such means, it will be possible for the electrodes to be reusable or disposable. Moreover, although the means here for determining an angular acceleration of the receptacle comprise an accelerometer, the determining means may not comprise a sensor but instead means for calculating the angular acceleration of the receptacle by derivation of the angular position data provided by the means for determining an angular displacement of the receptacle relative to the frame. In this case, it will be possible that said calculating means are not supported by the frame but instead are integrated in the remote control unit. The means for determining a torque exerted on the receptacle may comprise a torque meter instead of a force sensor. Said means for determining a torque may comprise neither a handle nor a force sensor linked to the handle. The means for determining an angular displacement may comprise a goniometer instead of an angular position sensor.

Similarly, it will be possible for the receptacle to have a shape other than that described. It will be possible, for example, for the receptacle to have a bottom wall connecting the two wings and the base of the stirrup.

Instead of a strap, the immobilizing means may comprise a cover that goes over the top of the receptacle in such a way that the assembly formed by the receptacle and by the cover will form a shoe that is open at the heel. This familiar shoe shape will also be reassuring for the patient especially since, apart from the cover, the patient will be able to see his foot and his ankle throughout the measurements.

Furthermore, although the means here for limiting the angular course of the receptacle are removable, it is conceivable that these means are not removable. Whether said means are removable or not, it will also be possible for said means to be movable relative to the frame in such a way as to modify the definition of the second position of the receptacle with respect to the frame. It will thus be possible to adapt the second position for each subject.

Although the device here has a panel rigidly connected to the frame and comprising means for height adjustment, such as adjustable feet, it will be possible for the device to have no panel, the frame being directly arranged on a support or on a panel that is independent of the device. Alternatively, it will be possible for the device to have a panel which is not rigidly connected to the frame but on which the frame rests. It will also be possible for the panel to have means for temporary connection to the frame.

It will be possible for the management means to have means for the storage of data processed by the processing means.

The locking means may differ from what has been described. For example, the first lever and the second lever may not extend perpendicularly with respect to each other.

The receptacle may be removable. It will thus be possible to replace the receptacle with another receptacle of a size and/or shape more suited to the subject concerned. For example, it is possible to envision a first size of receptacle for adults and a second size of receptacle for children. In the case of a receptacle for children, the receptacle may not completely conceal the various elements supported by the frame, on account of the lower height of the latter. Nonetheless, despite everything, the various elements will be arranged behind the receptacle, and therefore the frame will not be upsetting to children.

The frame may comprise a member for actuating the receptacle, for example a motor for motorized movement of said receptacle. Preferably, said member will then be able to be temporarily deactivated such that the receptacle remains able to be moved manually by the subject or by a medical person in order to perform other types of tests.

Still with a view to making the frame as simple as possible in order to reassure the subject, it will be possible for the frame to have a cover extending between the two side walls of the frame above the floor, which will make it possible to cover the various elements arranged behind receptacle. It will be possible for the cover to be mounted pivotably on one of the side walls so that, despite everything, it is still possible to access said various elements. It will be possible for the frame to have a larger number of walls in order to cover the various elements of the frame so that only the receptacle is visible to the subject. Alternatively, with the exception of the receptacle, the whole frame will be protected by an enclosure. Thus, only the receptacle will be visible to the subject.

1. A portable device for measuring physical parameters associated with the ankle joint of a subject, said device having:
a portable frame which has an open receptacle intended to receive a foot of the subject, the receptacle being mounted pivotably on the frame about a given rotation axis in such a way as to be arranged at the end of the frame with an opening of the receptacle directed toward the outside of the device and in such a way that the rotation axis coincides with the articulation axis of the subject's ankle when the subject's foot rests in the receptacle, and which has means for temporarily locking the receptacle in a first position,
means for determining a torque exerted on the receptacle,
means for determining an angular displacement of the receptacle relative to the frame,
means for determining an angular acceleration of the receptacle during an angular displacement of the receptacle,
at least the locking means and the means for determining a torque being arranged in the frame behind the receptacle when the opening of the receptacle faces the subject.

2. The device as claimed in claim 1, in which the locking means are arranged in such a way that, in the first position, a main wall of the receptacle, on which wall the sole of the foot is intended to rest, extends perpendicularly with respect to the floor of the frame.

3. The device as claimed in claim 1, in which the frame has a member for activating the locking means in such a way that, when the activating member is not powered, the locking means are active and, when the activating member is powered, the locking means do not lock the receptacle, which is able to pivot relative to the frame.

4. The device as claimed in claim 3, in which the locking means comprise a first lever which is mounted pivotably on the receptacle and which comprises a hook at its free end, the locking means additionally having a second lever of which one of the ends is mounted pivotably on the frame and comprises a hook for cooperating with the hook of the first lever in order to lock the receptacle in the first position, the second lever additionally being connected at its second end to the activating member in such a way that, when the activating member is not powered, the second end is also rigidly connected to the frame via the activating member.

5. The device as claimed in claim 3, in which the activating member is of the electromagnetic type and has a first portion rigidly connected to the frame and a second portion carried by the second end of the second lever, which portions disengage when the activating member is powered.

6. The device as claimed in claim 1, additionally having means for limiting an angular course of the receptacle between the first position and a second position.

7. The device as claimed in claim 6, in which the means for limiting an angular course of the receptacle are removable.

8. The device as claimed in claim 6, in which the limiting means are arranged in such a way that, in the second position, a main wall of the receptacle, on which wall the sole of the foot is intended to rest, extends at most 45 degrees from a floor of the frame.

9. The device as claimed in claim 1, in which the receptacle is configured as a bracket comprising two wings and a base from which the two wings extend, the base forming a main wall of the receptacle, on which wall the sole of the subject's foot is intended to rest.

10. The device as claimed in claim 1, additionally having a panel which comprises height-adjustable feet and on which the frame rests.

11. A portable frame of the device as claimed in claim 1, the frame having an open receptacle intended to receive a foot of the subject, the receptacle being mounted pivotably on the frame about a given rotation axis in such a way as to be arranged at the end of the frame with an opening of the receptacle directed toward the outside of the device and in such a way that the rotation axis coincides with the articulation axis of the subject's ankle when the subject's foot rests in the receptacle, the frame having means for temporarily locking the receptacle in a first position.
